(19) United States
(12) Patent Application Publication
(10) Pub. No.: US 2015/0182481 A1
(43) Pub. Date: Jul. 2, 2015

(54) USE OF POLYMER D-LACTIC ACID (PDLA) TO TREAT PAIN

(71) Applicant: Joel Steven Goldberg, Hillsborough, NC (US)

(72) Inventor: Joel Steven Goldberg, Hillsborough, NC (US)

(21) Appl. No.: 14/167,028

(22) Filed: Jan. 29, 2014

Related U.S. Application Data
(60) Provisional application No. 61/922,339, filed on Dec. 31, 2013.

(51) Int. Cl. A61K 31/194

(52) U.S. Cl. CPC A61K 31/194

(57) ABSTRACT

L-Lactate is required as a fuel for the continuous firing of nociceptors that produce many of the manifestations of chronic pain. Polymer D-lactic acid (PDLA) forms a spontaneous non-enzymatic, thermodynamically favored stereocomplex with sequestration of l-lactate or “trapping” of l-lactate. Injection or topical application of PDLA can reduce pain by decreasing the l-lactate available to activated nociceptors without sequestering lactate in non-disrupted nerves. C fibers and unmyelinated visceral afferents are most sensitive to the analgesia produced by PDLA. PDLA can be used to ameliorate pain from a wide variety of medical conditions including skin ulcers, wounds and burns; causalgia, radiculopathy, neuropathy; arthritis; and cancer. PDLA oligomers can be inexpensively produced from d-lactic acid in a home microwave. The predicted toxicity of PDLA is low and topical application or injection into perineural tissues for relief of pain may be very safe.

### CROSS-REFERENCES TO RELATED APPLICATIONS

This application claims the benefits of U.S. Provisional Patent Application No. 61/922,339 filed Dec. 31, 2013 each of which is incorporated herein by reference in its entirely.

### STATEMENT REGARDING FEDERALLY SPONSORED RESEARCH OR DEVELOPMENT

Not applicable

### BACKGROUND OF THE INVENTION

L-Lactic acid (lactic acid) is an end product of glucose metabolism and exists dissociated in the tissues in the form of l-lactate (lactate) and hydrogen ion. Recent evidence supports that lactate has an independent function to regulate neuronal activity, serve as a modulator of neurotransmission, promote inflammation by signaling of macrophages and most importantly serve as an energy source. (Immke & McCleskey, 2001; Philp, Macdonald, & Watt, 2005) These functions are separate from the changes in pH associated with lactic acid. (Stahl & Longhurst, 1992) The brain contains considerable quantities of lactate that can also be used as a substrate for the production of ATP when glucose is in short supply. (Schousboe et al., 1997) Astrocytes are known to manufacture and shuttle lactate to neurons for this purpose. (Schousboe, et al., 1997) In 1932, Feng showed that addition of lactate improved a peripheral nerve's capacity to function under the stresses of repeated stimulation after nerves were poisoned with iodoacetic acid. (FIG. 1) (Feng, 1932) His nerve preparation was an intact sciatic nerve that exhibited sustained neural activity. This sustained activity may be analogous to the peripheral nerve activity in patients who suffer from chronic pain.

Stahl and Longhurst studied the effects of lactic acid on ischemic visceral afferents nerves. (FIG. 2) (Stahl & Longhurst, 1992) They concluded that ischemic visceral afferents responded to lactic acid rather than to lactate or hydrogen ions or changes in PCO₂. Immke and McCleskey showed that lactate dramatically increases the activity of acid-sensing ion channels (ASIC) located on sensory neurons that innervate the heart. (Immke & McCleskey, 2001) It has not been proven whether this observation is explained by lactic acid or lactate interfering with ASIC or TRP channels. In contrast to prior art, in this invention, neural deprivation of lactate as a fuel source required for repetitive nociceptor activity is the primary mechanism of polymer d-lactic acid (PDLA) analgesia.

In 1988, Ikada described the spontaneous stereocomplex formed when d and l oligomers of lactic acid were combined. (Ikada, Jamshidi, Tsuji, & Hyon, 1987) Since that time, Goldberg and Weinberg have provided enzyme and HPLC evidence that a stereocomplex is formed when oligomers of PDLA are combined with l-lactate and described this reaction as sequestering or “trapping” lactate. (Goldberg, 2011; “USE OF POLYMER D-LACTIC ACID (PDLA) OR EQUIVALAENTS THEREOF TO INHIBIT GROWTH OF CANCER CELLS AND DIAGNOSE CANCERS,” 2012) This phenomena is believed to occur by chiral forces that are a component of Van der Waals forces. In this invention, PDLA traps lactate in the vicinity of nerves thereby inhibiting repetitive nociceptor activity and produces analgesia.

Injection and topical application of steroids, local anesthetics and viscous agents are common techniques utilized in the field of pain management. Oligomers of PDLA exist as hydrogels that slowly dissolve and can be injected or topically applied near sites that generate pain. PDLA primarily produces analgesia through a thermodynamically favored stereocomplex reaction with l-lactate that can reduce free lactate at sites in the vicinity of nociceptors.

### DESCRIPTION OF THE FIGURES

FIG. 1 shows the beneficial effect of sodium lactate on a nerve poisoned by iodoacetic acid (I.A.A.) and continuously tetanized: (Feng, 1932) The x axis is time in minutes and the y axis is sustained galvanic contractions. Line A is sustained galvanic contractions by I.A.A. solution alone: Line B is sustained galvanic contractions by I.A.A. solution containing lactate in concentration 180 mg/100 cc. Lactate enhances repeated nerve tetanization. (Feng, 1932)

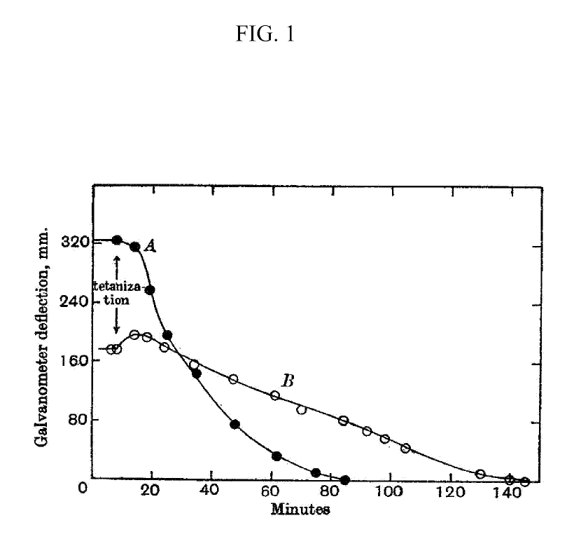

FIG. 2 shows the increase firing of C fibers after injection of lactic acid as a neural histogram and representative neurograms. (Stahl & Longhurst, 1992) Histogram labeled A is during 5 minutes of ischemia, histogram labeled B is during 5 minutes of 12% hypercapnia and histogram C is during 300 mM lactic acid injection. The representative neurograms are labeled lowercase letters (a-f) and arrows in the histograms of A, B, C indicate periods of representative neurograms. (Stahl & Longhurst, 1992)

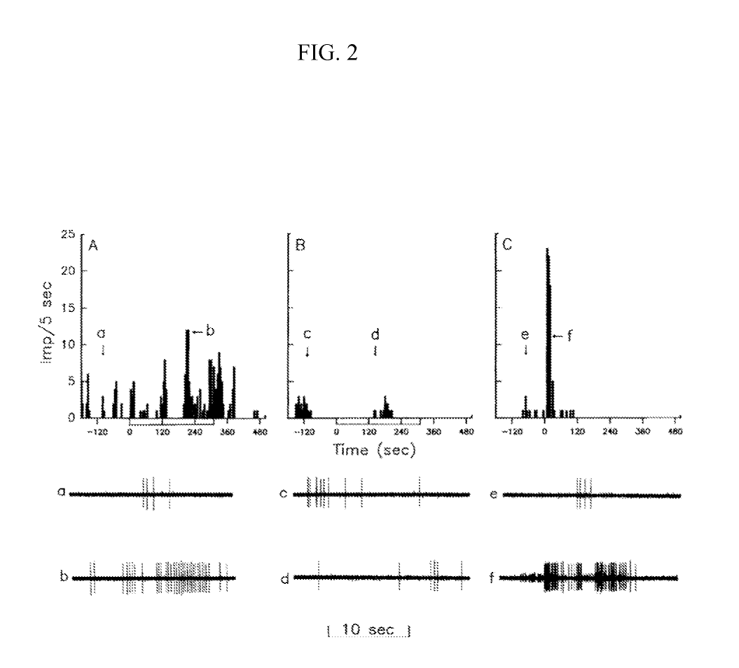

FIG. 3 is a cross section of a peripheral nerve. Unless there is disruption of the nerve, it is unlikely that PDLA applied in the vicinity of a peripheral nerve labeled 1 will diffuse to the nociceptors within the endoneurium labeled 4. The epineurium is labeled 2 and the perineurium is labeled 3. Therefore, application of PDLA to non-disrupted nerves is unlikely to sequester lactate in the vicinity of nociceptors or other nerve axons.

### DETAILED DESCRIPTION OF THE INVENTION

Select oligomers of PDLA are known to complex lactate, the reaction of which is spontaneous, thermodynamically favored and non-enzymatic. Lactate is an essential agent that provides for continuous long term transmission of a nerve and more specifically nociceptors. (Feng, 1932; Immke & McCleskey, 2001; Stahl & Longhurst, 1992) Continuous transmission of a nociceptor is a hallmark of chronic pain.

Oligomers of PDLA exist as hydrogels of low toxicity that can be injected or applied topically near sites of nociceptive or neuropathic pain. Some of these sites that are commonly associated with inflammation include skin ulcers, joints, tendons and muscles or sites of nerve injury. Another important site includes areas that contain neural tissue within the vicinity of malignant tumors.

Injections and topical application of steroids, local anesthetic and viscous agents are common techniques utilized in the field of pain management. Often these injections are performed with fluoroscopic, ultrasound or nerve stimulation guidance however because PDLA is water soluble and of predicted low toxicity, injections without imaging may be possible at a substantially reduced cost. At room and body temperature PDLA exists as a hydrogel that slowly hydrolyzes and therefore PDLA injections or topical applications provide pain relief for periods of time, much greater than the present agents in prior art including steroids, local anesthetics or viscous agents.

### Mechanism of Action

Stereocomplex Reaction of PDLA with l-Lactate and Chiral Forces

Prior art has largely recognized the significance of stereocomplex reactions to form new materials in the field of polymer chemistry. However, PDLA also serves as a template for binding l-lactate. The dramatic change in melting point (approximately 60 C.°) when PDLA is mixed with PLLA to form a stereocomplex and the formation of a stereocomplex when l-lactate reacts with PDLA suggests that a strong chiral attractive force which is a component of Van der Waals forces exists between these enantiomers. In racemic mixtures these chiral forces between enantiomers may not normally be observed because charge forces or hydrogen bonding predominate. However, when negatively charged PDLA reacts with negatively charged l-lactate the chiral force overcomes the repelling charge and hydrogen bond forces; thus a stereocomplex is formed. The formation of this stereocomplex can occur if charge and hydrogen bond forces are decreased over distance along the polymer units such that the hydrophobic “tail” of PDLA can template molecules of l-lactate. Since most organisms have evolved with l enantiomers of amino acids and d enantiomers of glucose, chiral polymers could be future drug targets.

Lactate Trapping

Goldberg and Weinberg have provided enzyme and HPLC evidence that a stereocomplex is formed when oligomers of PDLA are combined with l-lactate and described this reaction as sequestering or “trapping” lactate. (Goldberg, 2011; “USE OF POLYMER D-LACTIC ACID (PDLA) OR EQUIVALENTS THEREOF TO INHIBIT GROWTH OF CANCER CELLS AND DIAGNOSE CANCERS,” 2012) Lactate serves many functions in addition to being a waste product of glycolysis. Lactate shuttling occurs between cancer cells and between astrocytes and neurons in the central nervous system. (Schousboe, et al., 1997) Parasites such as Plasmodium produce large quantities of lactate. Trapping lactate with PDLA is an extraordinary versatile chemical reaction that may have medical uses in the treatment of cancer, malaria and in this invention, pain and also non-medical uses. In this invention, lactate trapping produces analgesia by disrupting the energy source required by nociceptors to continuously transmit information via action potentials to the brain.

Lactate as a Fuel for Nociceptors

Normally nociceptors exist nascent in equilibrium with their environment such that resting membrane potentials are maintained without generation of an action potential. When the environment changes, the equilibrium is disrupted, and membrane permeability changes produce an action potential. The information within the action potential is transmitted to the spinal cord (where modulation occurs) and to higher center in the brain and perceived as pain. The generation of an action potential requires energy initially supplied by glucose but repetitive stimulation requires that the nerve shift energy substrates to lactate. This shift in energy substrate is well established in the central nervous system where astrocytes shuttle lactate to neurons. (Schousboe, et al., 1997) Lactate then becomes the preferred energy substrate for nociceptors. Lactate can be converted to pyruvate within the nerve by lactate dehydrogenase 1 (LDH1). LDH1 is the predominant LDH isoform within nerves and unlike LDH5 it preferentially converts lactate to pyruvate. (Bittar, Charnay, Pellerin, Bouras, & Magistretti, 1996) Pyruvate can be converted to Acetyl-coA that the nerve can utilize to produce ATP. This mechanisms explains the results of Feng reported 82 yrs ago. (Feng, 1932) Stereocomplexes of PDLA with l-lactate is a thermodynamically favored, non-enzymatic reaction that traps l-lactate. When lactate is not available as a fuel to maintain continuous generation of action potentials the nociceptors cannot effectively transmit information and pain is reduced.

### Properties of PDLA

pKa

The various oligomers of PDLA have a pKa that closely approximates 3.86 that of lactic acid. At pH 7.4 the microspecies of oligomers are predominantly in the charged form:

pH=pKa+log [PDLA⁻]/[PDLA]

7.4=3.86+log [PDLA⁻]/[PDLA]

3,467=[PDLA⁻]/[PDLA]

For oligomers of low molecular weight, such as n=2 units, charge predominates over chiral forces inhibiting stereocomplex formation. However as the size of the oligomers increase charge considerations become less important and the “tail” of the PDLA molecule approaches neutral to allow PDLA to sequester lactate. For topical or local injection therapy in the vicinity of a nerve, oligomer size is less important than for systemic injection. Systemic administration of PDLA requires that PDLA diffuse across vascular endothelium to reach nociceptors and molecular weight impedes this diffusion.

Routes of Administration

Topical

In this invention, PDLA is applied topically or injected in the vicinity of nerves. When applied topically such as for the treatment of pain from skin ulcers, wounds or burns, PDLA immediately contacts nociceptors.

Parental

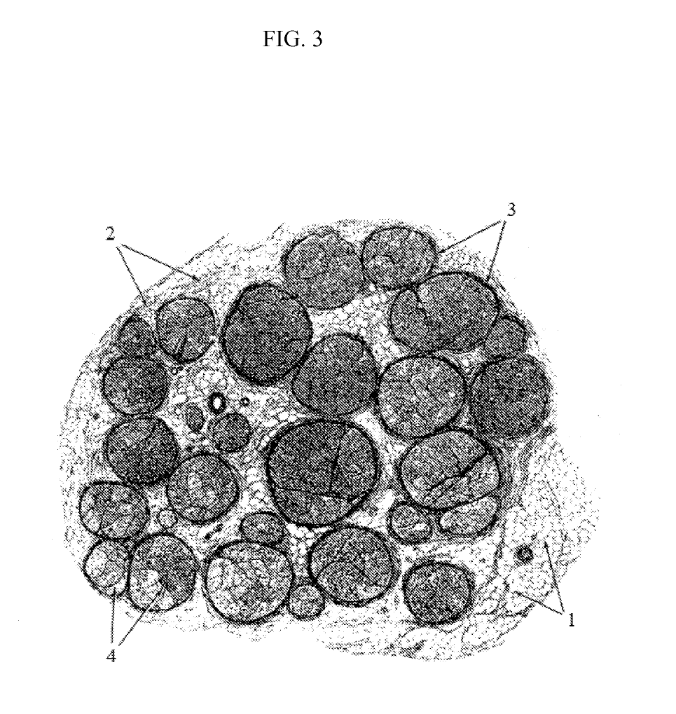

When injected into the vicinity of a disrupted nerve, PDLA can diffuse through the disrupted perineural tissues, epineurium, perineurium, endoneurium and reach the axons of nociceptors that can be C fibers, visceral afferents or Aδ fibers. PDLA can then complex lactate in the vicinity these nociceptors. Examples of use would be for relief of pain from causalgia, radiculopathy, arthritis and cancer. In non-disrupted nerves, it would be unlikely that PDLA could cross the various tissues to trap lactate. (FIG. 3) Therefore, PDLA would not trap lactate in the vicinity of normal nerves and not impair normal nerve function.

Systemic Administration

Oligomers of PDLA likely to have drug like properties according to Lipinski's criteria and diffuse from vasa nervorum to axons are listed in Table 1. The bioavailability and effectiveness of systemic administration of PDLA for the treatment of pain is not known.

TABLE 1
Oligomers of PDLA with predicted drug like properties for
systemic administration

| # of d-lactate | Molecular | Hydrogen bond | Hydrogen bond |
|---|---|---|---|
| monomers | weight | acceptors | donors |
| 2 | 162 | 5 | 1-2 |
| 3 | 234 | 7 | 1-2 |
| 4 | 306 | 9 | 1-2 |
| 5 | 378 | 11 | 1-2 |
| 6 | 460 | 12 | 1-2 |

PDLA Metabolism and Excretion

PDLA and PDLA—lactate stereocomplexes are expected to slowly dissolve in the body through ester hydrolysis in a similar manner as stereocomplexes of PLLA-PDLA (polylactides). Polylactides are used in the manufacture of cardiac stents, drug delivery systems, prosthetic devices and wound dressings and are degraded into d and l lactate. L-lactate is a natural product of glycolysis and can be metabolized or converted into glycogen in the liver. D-lactate can be excreted in the urine or sweat.

Toxicity of PDLA

The toxicity of PDLA in humans or animal has not been reported but the stereocomplex formed by PDLA and PLLA (PDLL or polylactide) is found in prosthetic devices, cardiac stents, biodelivery systems and wound coverings. (Panyam & Labhasetwar, 2004; Tamai et al., 2000) A metabolite of PDLA, d-lactic acid, is neurotoxic at average plasma concentrations of 7.98 mM with reversible symptoms of altered mental status, dysarthria, and ataxia. (Uribarri, Oh, & Carroll, 1998) Measurement of plasma d-lactate in asymptomatic exercising subjects has been reported as 12 mM. (Kondoh, Kawase, & Ohmori, 1992) These seemingly high plasma concentrations of d-lactate are possible because the overwhelmingly predominant microspecies of PDLA is charged and unlikely to cross the blood-brain-barrier at pH of 7.4.

Costs

D-lactic acid, PDLA and possible synthetic precursers are moderately expensive reagents. Table 2 list some known suppliers with costs:

TABLE 2
Cost of PDLA or reagents to synthesize PDLA

| Supplier | Agent | Quantity | Cost (USD) |
|---|---|---|---|
| Sigma-Aldrich | D-lactic acid | 100 mg | 187.50 |
| Sigma-Aldrich | Sodium d-lactate | 1 g | 90.70 |
| Santa Cruz Bio. | Sodium d-lactate | 1 g | 90.00 |
| Sigma-Aldrich | PDLA | 100 mg | 87.50 |
| | MW~124,000 | | |

Synthesis of specific molecular weight oligomers requires the expertise of an experienced organic chemist. However, mixed oligomers of PDLA that have demonstrated analgesic activity can be prepared from d-lactic acid in a home microwave oven. (Pandey & Aswath, 2009)

Benefits to Society

Chronic pain precipitated by age, trauma, malignant or non-malignant disease usurps quality of life. The famous quote by Albert Schweitzer “Pain is a more terrible lord of mankind than even death itself” succinctly summarizes the plight of humans unfortunate to suffer chronic pain. Chronic pain is more complex than a nociceptive disturbance, but of all the causes and possible treatments of chronic pain, interrupting the firing of nociceptors either through medications, injections, stimulation or behavioral therapies has shown promise in ameliorating the symptoms. By formation of stereocomplexes, lactate, a known, but not well recognized substrate essential for repetitive neural transmission, can be trapped by PDLA. PDLA can be prepared in a home microwave. If toxicity studies support the use of PDLA, PDLA has the potential to improve the lives of millions suffering chronic pain at a price society should be able to afford.

### Experimental Data

A 0.5×1.5 cm skin lesion of approximate depth of 1.5 mm was made in the left index finger of a subject (jsg) with 60 grit sandpaper. The lesion produced vigorous bleeding and pain. Application of approximately 10 mg of PDLA in the form of a hydrogel to the lesion produced an intense burning lasting approximately 30 seconds after which there was complete analgesia without loss of sensation (numbness). The analgesia lasted 24 hours until the lesion was exposed to water. Additional PDLA was applied and analgesia continued thereafter until the lesion completely healed in 10 days.

### REFERENCES

Bittar, P. G., Charnay, Y., Pellerin, L., Bouras, C., & Magistretti, P. J. (1996). Selective distribution of lactate dehydrogenase isoenzymes in neurons and astrocytes of human brain. J Cereb Blood Flow Metab, 16(6), 1079-1089.
Feng, T. P. (1932). The role of lactic acid in nerve activity. J Physiol, 76(4), 477-486.
Goldberg, J. S. (2011). Stereocomplexes Formed From Select Oligomers of Polymer d-lactic Acid (PDLA) and l-lactate May Inhibit Growth of Cancer Cells and Help Diagnose Aggressive Cancers-Applications of the Warburg Effect. Perspect Medicin Chem, 5, 1-10.
Ikada, Y., Jamshidi, K., Tsuji, H., & Hyon, S. H. (1987). Stereocomplex Formation between Enantiomeric Poly(Lactides). Macromolecules, 20(4), 904-906.
Immke, D. C., & McCleskey, E. W. (2001). Lactate enhances the acid-sensing Na+ channel on ischemia-sensing neurons. Nat Neurosci, 4(9), 869-870.
Kondoh, Y., Kawase, M., & Ohmori, S. (1992). D-lactate concentrations in blood, urine and sweat before and after exercise. Eur J Appl Physiol Occup Physiol, 65(1), 88-93.
Pandey, A., & Aswath, P. B. (2009). Microwave synthesis of poly(L-lactic acid). J Biomater Sci Polym Ed, 20(1), 33-48.
Panyam, J., & Labhasetwar, V. (2004). Targeting intracellular targets. Curr Drug Deliv, 1(3), 235-247.
Philp, A., Macdonald, A. L., & Watt, P. W. (2005). Lactate—a signal coordinating cell and systemic function. J Exp Biol, 208(Pt 24), 4561-4575.
Schousboe, A., Westergaard, N., Waagepetersen, H. S., Larsson, O. M., Bakken, I. J., & Sonnewald, U. (1997). Trafficking between glia and neurons of TCA cycle intermediates and related metabolites. Glia, 21(1), 99-105.
Stahl, G. L., & Longhurst, J. C. (1992). Ischemically sensitive visceral afferents: importance of H+ derived from lactic acid and hypercapnia. Am J Physiol, 262(3 Pt 2), H748-753.
Tamai, H., Igaki, K., Kyo, E., Kosuga, K., Kawashima, A., Matsui, S., et al. (2000). Initial and 6-month results of biodegradable poly-l-lactic acid coronary stents in humans. Circulation, 102(4), 399-404.
Uribarri, J., Oh, M. S., & Carroll, H. J. (1998). D-lactic acidosis. A review of clinical presentation, biochemical features, and pathophysiologic mechanisms. Medicine (Baltimore), 77(2), 73-82.
USE OF POLYMER D-LACTIC ACID (PDLA) OR EQUIVALAENTS THEREOF TO INHIBIT GROWTH OF CANCER CELLS AND DIAGNOSE CANCERS. (2012). USA Patent No. US 2012/0195850 A1.

Having described my invention, I claim:

1. A method to treat pain by administration of Poly-D-Lactic Acid (PDLA).

2. The method of claim 1 where PDLA is administered parentally.

3. The method of claim 1 where PDLA is administered topically.

4. The method of claim 1 where administration of PDLA is in the form of an oral prodrug of PDLA.

5. A method to treat pain by administration of oligomers of Poly-D-Lactic Acid (PDLA) where PDLA is comprised of oligomers that weigh less than 500 daltons.

6. The method of claim 5 where administration of oligomers of PDLA are in the form of an oral prodrug of PDLA.

7. The method of claim 5 where oligomers of PDLA are administered topically.

8. The method of claim 5 where oligomers of PDLA are administered parentally.
